(19) United States
(12) Patent Application Publication
(10) Pub. No.: US 2010/0322976 A1
(43) Pub. Date: Dec. 23, 2010

(54) TONIC AND FOOD SUPPLEMENT COMPRISING ALOE VERA AND HONEY

(76) Inventor: Kuldeep Sharma, Auckland (NZ)

Correspondence Address: YOUNG & THOMPSON, 209 Madison Street, Suite 500, Alexandria, VA, 22314, US

(21) Appl. No.: 12/525,946

(22) PCT Filed: Feb. 5, 2008
(86) PCT No.: PCT/NZ2008/000015

§ 371 Date: Aug. 10, 2009

(30) Foreign Application Priority Data
Feb. 5, 2007 (NZ) 553016

(51) Int. Cl. A61K 35/64; A61P 29/00; A61P 37/04

(52) U.S. Cl. 424/278.1; 424/537

(57) ABSTRACT

A mixture of honey (preferably manuka honey) with aloe vera extract is concentrated for distribution and then diluted before use as a tonic or food supplement for mammals, particular those with compromised or poorly performing immune systems. In particular (though without limit) the composition is useful in the control of chronic mastitis in dairy cows.

### FIELD

This invention relates to a composition useful as a tonic and a food supplement, made from naturally occurring ingredients.

### BACKGROUND

Use of the plant aloe vera (and various species and subspecies thereof) in the prophylaxis and treatment of various diseases and injuries of man and animals has become less popular since the advent of “magic bullet” pharmacology, such as the discovery of antibiotics and other pharmaceuticals having relatively specific actions. The plant is included in the crest of the Royal Veterinary College (London). Nowadays this type of medication is generally regarded as part of the “alternative medicines” category, although it is accepted as a beneficial treatment for such as wounds and burn injuries in humans, for example. Aloe vera is one of a group of phytochemicals that can selectively modulate cells of the immune system (Tan & Vanitha, Curr Med Chem 2004 Jun. 11(11) 1423-30).

### LITERATURE

For example, the book “Aloe Vera Nature's Gift” by David Urch. Bristol: Blackdown Publications (ISBN 0 9536569 0 X 1999) describes use of aloe vera extracts in the treatment of a number of animal diseases.

Also, the book “Creatures in our care: the veterinary uses of Aloe Vera” by Bill C Coats and Richard E Holland (1985) (published in the USA by Coats & Holland) describes use of aloe vera extracts in the treatment of a number of animal diseases.

Regarding the disease of bovine mastitis, Urch describes the use of intramammary infusions of aloe vera as follows: 1. Strip the affected quarter. 2. Infuse a mixture of 20 ml aloe vera spray solution and 10 ml aloe vera gelly into the quarter. 3. Massage the quarter with aloe vera gelly. 4. Repeat twice daily. 5. Also feet the affected cow with 250 ml aloe vera either into the food or [as a drench]. 6. Continue until symptoms are resolved.

Coats & Holland also describe trials of aloe vera in relation to mastitis in cows, with a method as follows: (1) injection of 50 ml “Aloe Activator” intravenously on day 1; injection of 25 ml “Aloe Activator” intramuscularly on days 2 and 3, and optionally, 20 ml of “Aloe Activator” intramammary after the affected quarter has been stripped out. A more preferred method comprises repeated intramammary infusion of “Aloe Activator”. A trial by a farmer, comprising adding aloe vera juice to the feed of a herd of 60 cows, resulted in 400 lb more milk per day. The use of controls was not particularly evident.

None, of the above experiments, nor those in the patent literature cited below mixed honey with the aloe vera compositions.

Another development in alternative therapies has added to the antibacterial effects of any form of honey with manuka honey: following the discovery of special properties in at least some examples of honey made from the flowers of the manuka tree (Leptospermum spp) which is endemic in New Zealand. (Any honey will be a mixture: manuka honey is predominantly from manuka flowers). An as yet uncharacterised substance, called by the discoverer Prof. P Molan of Waikato University, the “UMF factor” supplements the osmotic, peroxide content, and enzymic antibacterial properties of conventional honey. The UMF factor has a molecular weight of approximately 500 and is heat-stable to a temperature higher than that which degrades the enzymes. The UMF rating of a particular batch of honey is measured in a standard test by the distance to which diffusion from a well through an agar plate bearing a coating of bacteria succeeds in inhibiting growth of the bacteria. There are many publications from the Molan group.

A trial carried out by Jane Lacy-Hulbert of Dexcel, New Zealand and reported in June 2005 (Dexcelink, winter 2005) compared intramammary infusions of manuka honey against antibiotic therapy (Lincocin forte) or no treatment in cows having a high somatic cell count (indicating chronic, low-grade mastitis). The honey treatment, administered six times, gave a slightly lower cure rate to the antibiotic option for infections of Streptococcus uberis, but was about half as useful (in terms of bacteriological cure rate) for Staphylococcus aureus or Streptococcus dysgalactiae. Nevertheless a non-antibiotic treatment of honey does not require that a withholding period be imposed on milk from any of the quarters subsequent to treatment.

Subclinical mastitis is frequently found in dairy cows and is a bacterial infection of the mammary gland caused often by S aureus, S uberis, and less commonly Escherischia coli and other micro-organisms. These organisms are likely to carry antibiotic resistant genes. Factors encouraging the persistence of mastitis include poor general health, poor milking hygiene, and badly operated milking machinery. The bulk milk supply shipped by a farm to a dairy factory carries an indication of chronic or acute mastitis. It is likely to contain a high level of white blood cells; principally neutrophils, giving rise to a measure called the somatic cell count. If over 450,000 cells per ml, the milk is graded down under New Zealand rules. If under perhaps 150,000 the milk is of relatively high quality and the farmer is likely to be paid a premium. Therefore there is an economic benefit (quite apart from the cow's position) from alleviating chronic mastitis by use of aloe vera type medications; preferably by a prophylactic food additive route and preferably with an “enhancing agent” if one is available.

### PATENT LITERATURE

Hassler et al (U.S. Pat. No. 5,846,543) describe successful treatment of bovine mastitis by intramammary injection of 10.5 ml aloe vera juice, plus 0.5 ml Echinacea extract, 0.5 ml “Wild Ginseng Supreme” and 0.5 ml of a mixture of gelsemium, pokeroot and aconite; twice daily over from three to five days. How treatment was assessed is not described. Hassler et al in US2008020061 describe a cream for topical application, for treatment of bovine mastitis, which predominantly includes aloe vera. The inventors assessed treatment by the Somatic Cell Count, which showed a fall from 5.5 million to, eventually, 91,000. WO99/13892 Laboratorios Pisa (MX) describes an antimastitic composition and its preparation for treatment of mastitis in domestic animals. The composition includes juice or gel of aloe vera, (predominating at about 12-15%) along with other herbal remedies, salts, etc. These inventors believe that immunomodulation is one component of the action.

### PROBLEM TO BE SOLVED

A particular problem to be solved is to provide a suppression or preferably a cure of mastitis in dairy animals, using a composition that is effective yet does not include conventional antibiotics, so that it can safely be administered without deleterious effects such as causing antibiotic resistance. More generally, the problem is to provide an orally administered tonic that has a generally beneficial effect on an animal's resistance to disease; perhaps expressed as a raised activity of the immune system.

### OBJECT

The object of this invention may be stated as to provide an improved tonic or food supplement for humans or animals, or a prophylactic (oral or topical) or a therapeutic (topical or intramammary) treatment for disease, or at least to provide the public with a useful choice.

### STATEMENT OF INVENTION

In a first broad aspect this invention provides a mixture of aloe vera and honey, the composition being sold in a concentrated form for distribution then use as a food supplement or tonic for animals including humans.

Preferably the honey is a predominantly manuka honey

Optionally the manuka honey has a known UMF (specific antibacterial) rating.

In a related aspect this invention provides a mixture of aloe vera and manuka honey, the composition being in a non-concentrated form ready for oral administration.

In a yet further aspect this invention provides a mixture of aloe vera and manuka honey, the composition being in a form ready for use by intramammary administration suitable for the treatment of mastitis in cows.

In a second broad aspect this invention comprises a supplement/tonic composition comprising or including on a weight for weight basis the following ingredients

| | |
|---|---|
| Manuka Honey | from 6% to 10%, |
| aloe vera powder | from 79% to 98.7% |
| Sodium Benzoate | from 0.1% to .5% |
| Potassium sorbate | from 0.1% to .5% |
| Ginger powder | from 2% to 8% (flavouring) |
| pH adjuster: | citric acid 0.1% or Apple Cider Vinegar 0.1%. |

The preferred composition has a pH in the range of 3.5 to 5, preferably 4.

Preferably the composition also includes tea tree oil or extracts.

Preferably the composition also includes garlic powder or extracts.

Preferably the said composition also includes Azadirachta indica (neem).

Preferably the said composition also includes Curcuma longa alias Amomun curcuma (turmeric)

Optionally the said composition includes fruit flavours, or the aforementioned ginger powder.

(The aloe powder may be freeze dried, or may be whole leaf spray dried powder)

In an alternative aspect said composition may comprise (w/w)

| | |
|---|---|
| Reconstituted aloe vera | 80% |
| Manuka honey | 10% |
| Sodium benzoate | 0.1% |
| Potassium sorbate | 0.1% |
| Cider vinegar | 0.1% |
| Ginger powder or extract | 2% |
| Tea tree oil | 2% |
| Azadirachta indica | 2% |
| Curcuma longa or Amomun curcuma | 2% |
| Garlic extract or powder | 2% |

Preferably the composition is concentrated to from 2:1 to about 20:1

Optionally the composition is freeze dried into a powder.

Optionally the composition is carbonated or oxygenated, as for use in a beverage.

In other forms the present invention consists in a food supplement comprising or including

| | |
|---|---|
| Reconstituted aloe vera | from 10% to 80% |
| Manuka honey | from 2% to 10% |
| Sodium benzoate | from 0.1% to 0.2% |
| Potassium sorbate | rom 0.1% to 0.2% |
| Cider vinegar | from 0.1% to 0.2% |
| Ginger powder or extract | from 2% to 5% |
| Tea tree oil | from 2% to 5% |
| Azadirachta indica | from 2% to 5% |
| Curcuma longa or Amomun curcuma | from 2% to 5% |
| Garlic extract or powder | from 2% to 5% |

In a third broad aspect the invention provides a method for reducing the incidence of chronic bovine mastitis, as evidenced by the somatic cell count in milk, comprising the prophylactic administration of a mixture including effective amounts of aloe vera and honey per day orally or in the food to an entire herd during the lactation season.

Preferably the honey is a predominantly manuka honey.

A preferred amount for prophylactic use is about 1 ml per cow per day (the quantity is expressed as the concentrate).

A more preferred dose is 3 to 5 ml per day.

An example preferred dosage for therapeutic use is three doses of 30 ml (expressed as concentrate) given orally over one week.

Optionally the mixture is given by intramammary injection—into the teat canal—after stripping the affected quarter, repeatedly over a period of time.

Optionally the same procedure is used in other dairy animals selected from the range of: dairy goats, milking ewes, buffaloes, camels and mares.

The present invention relates to a food supplement or food tonic for humans and animals that is made of natural ingredients that have a role in modulating the mammalian immune system.

### PREFERRED EMBODIMENT

The description of the invention to be provided herein is given purely by way of example and is not to be taken in any way as limiting the scope or extent of the invention.

Throughout this specification unless the text requires otherwise, the word “comprise” and variations such as “comprising” or “comprises” will be understood to imply the inclusion of a stated integer or step or group of integers or steps but not the exclusion of any other integer or step or group of integers or steps.

### FIGURES

FIG. 1: Graph of somatic cell counts against time, for a herd of 260-290 dairy cows for a period of 3.5 years. The data are derived from bulk sample tests.

### TABLES

Table 1. Effect of a short treatment according to the invention on somatic cell count in some chronic mastitis cases.

Table 2. Dose-response effects on somatic cell counts in randomly selected cows, over 16 weeks.

Table 3. Dose-response effects on feed intake and milk parameters.

Table 4: Bacterial growth inhibition.

### EXAMPLE 1

The invention generally comprises a mixture of honey and an extract of aloe vera, prepared for sale in a concentrated form, for use as a tonic and prophylactic mixture. Other applications, ranging from therapeutic applications through topical creams to beverages for consumption by humans may arise.

The presently preferred type of honey is manuka honey, although the inventor expects that any honey or components thereof may likewise exhibit synergism with the aloe vera component. Some examples of manuka honey include effective amounts of the “UMF factor” which depends largely on the composition of the flowers from which the bees collected pollen, and which is measured by an agar diffusion/bacterial growth inhibition test. It is believed that the combination of aloe vera and honey has a synergistic property in the treatment of at least chronic mastitis in dairy cows and high-UMF manuka honey has a greater synergistic effect. This specification emphasises treatment of chronic mastitis since there exists a quantitative test of severity (the somatic cell count), but the invention is applicable to a number of long-term conditions in which the immune system is believed to be depressed. In particular it should be noted that the dataset provided herein has not included details on the UMF content, if any, of the manuka honey in the invention.

The composition of one preferred mixture (1A) is as follows:

| | | |
|---|---|---|
| 1. | Reconstituted aloe vera | from 10% to 80% |
| 2. | Manuka honey | from 2% to 10% |
| 3. | Sodium benzoate | from 0.1% to 0.2% |
| 4. | Potassium sorbate | from 0.1% to 0.2% |
| 5. | Cider vinegar | from 0.1% to 0.2% |
| 6. | Ginger powder or extract | from 2% to 5% |
| 7. | Tea tree oil | from 2% to 5% |
| 8. | Azadirachta indica | from 2% to 5% |
| 9. | Curcuma longa | from 2% to 5% |
| 10. | Garlic extract or powder | from 2% to 5% |

The amount of components 6 to 10 in the above list (and especially 8=neem, and 9=turmeric) are as yet not fully assessed.

The composition of another preferred mixture (1B) is as follows:

| | | |
|---|---|---|
| 1. Honey | 6% | |
| 2. Aloe vera | 93.7% | (as dry weight) |
| 3. Sodium Benzoate | 0.1% | |
| 4. Potassium sorbate | 0.1% | |
| 5. Citric Acid 0.1% or Apple Cider | 0.1% | (to adjust pH to 3-5). |
| Vinegar | | |

The mixture is preferably concentrated during manufacture, so that the water content is reduced by from 50% to 95%. In 1B, the type of honey (flowers of origin) is unspecified, although manuka honey of an unspecified UMF rating has been used in the cited results. Should the UMF factor turn out to be important, each manuka honey batch used would have a UMF rating of at least 3 and possibly as much as 20.

### Preparation

The preferred composition is prepared by the following procedure:
1 50% water (de-ionized) is charged into a 2000 litre vessel.
2 Potassium sorbate and sodium benzoate is added with rapid mixing.
3 Aloe vera powder is added and fully dissolved with rapid mixing.
4 Manuka honey is added and mixed slowly into the mixture.
5 The pH level of the mixture is read.
6 Citric acid or cider vinegar is added to adjust the pH to between 3 and 5 pH units.

The product is filtered prior to filling in an appropriate container. Appropriate containers are plastic or glass. If the product is prepared for use as a beverage, it may be carbonated or oxygenated or sold as a concentrate.

### EXAMPLE 2

### Prophylactic Administration to a Herd of Dairy Cows

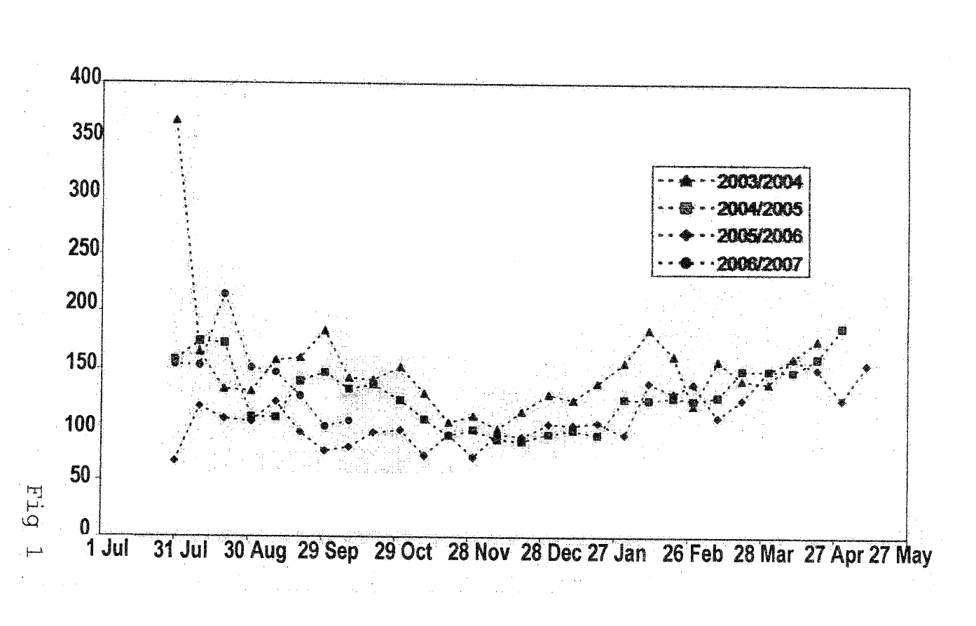

See FIG. 1. Somatic cell count (SCC) data for a herd of 260-280 dairy cows was collected over two years namely the 2003-2004 season and 2004-2005 season; and then the herd was fed the supplement for a further year and a half (2006-2007 and 2007 to date) at the rate of 1 or 2 mls daily (expressed as concentrate) by either drenching or by mixing the material into some food supplement that was totally consumed. The graph of FIG. 1 displays the somatic cell count of fortnightly bulk (all-herd) milk samples, in units of thousand cells per ml—so that the upper border of the graph (400,000 cells/ml) is still below the level at which the milk would be graded down (at 450,000 cells/ml). The horizontal axis is in time, the years being overlaid. The graph shows that in general the bulk milk somatic cell counts as determined by the dairy factory were lower during use of the prophylactic supplement, although in each year there is a common trend of the SCC to fall at or around the end of November. It will be noted that the circle and the diamond points (treatment) are generally lower than the squares and the triangles (pre-treatment).

The averaged SCC counts for 2003/4 are 146,000 cells/ml; for 2004/5 are 121,000 cells/ml; for 2005/6 are 104,000 cells/ml; for 2006/7 are 140,000 cells/ml (early part of season only). One must remember that just one or a few infected cows can cause the bulk SCC to rise significantly.

Note that since the composition is “Organic” according to the relevant industry definitions, no special care to avoid introducing milk from a herd receiving the dietary supplement into the human food chain is required. For instance, there are no antibiotic residues to cause allergies or to promote antibiotic resistance after ingestion.

### EXAMPLE 3

### Therapeutic Administration to Dairy Cows with Chronic Mastitis

Table 0.1 describes the use of the honey and aloe vera composition of the invention in 8 cases of chronic mastitis in identified cows. The individual-cow herd tests in February preceded treatment with three large doses of tonic (30 ml of concentrate-equivalent) administered orally over 1 week. The April tests show reduced SCC in 7 of 8 cows, and the May tests show reduced SCC in 7 of 8 cases also. As shown in FIG. 1, the natural biology of SCC is that it rises towards the end of lactation (end of May, approximately). The total herd average shows such a rise. The inventor concludes that the treatment has a marked beneficial effect.

TABLE 1
Effect of Tonic on Somatic Cell Count (,000)

| Cow Number | Herd Test Feb | Treatment | April | May |
|---|---|---|---|---|
| 15 | 1417 | y | 1002 | 1263 |
| 142 | 1262 | y | 839 | 696 |
| 88 | 3742 | y | 1385 | 524 |
| 87 | 457 | y | 292 | 505 |
| 95 | 1262 | y | 251 | 288 |
| 120 | 3085 | y | 92 | 120 |
| 75 | 453 | y | 24 | 54 |
| 19 | 208 | y | 311 | 141 |
| Total | 10916 | | 4196 | 3591 |
| Average | 1364 | | 524 | 448 |
| Total herd | 228 | | 166 | 215 |
| average | | | | |

In another experiment, different amounts of tonic were given orally for 16 weeks to four groups each of 3 Jersey cows, to determine if there was an optimum dose in terms of SCC control. As seen in Table 2, the 1 ml dose and the 5 ml doses have a small effect only as compared to the control (leftmost column), while the 10 ml regime (rightmost column) was effective immediately and maintained lower levels than any other dose during the experiment.

TABLE 2
effect of different amounts dosed.

Tonic

| | | 0 ml | 1 ml | 5 ml | 10 ml |
|---|---|---|---|---|---|
| Week | 1 | 280 | 280 | 280 | 200 |
| | 2 | 284 | 290 | 290 | 200 |
| | 3 | 280 | 285 | 285 | 210 |
| | 4 | 260 | 280 | 270 | 200 |
| | 5 | 250 | 260 | 260 | 190 |
| | 6 | 270 | 250 | 250 | 190 |
| | 7 | 280 | 260 | 250 | 190 |
| | 8 | 250 | 250 | 240 | 180 |
| | 9 | 240 | 230 | 240 | 180 |
| | 10 | 250 | 230 | 240 | 170 |
| | 11 | 260 | 220 | 230 | 170 |
| | 12 | 250 | 220 | 220 | 180 |
| | 13 | 240 | 190 | 190 | 170 |
| | 14 | 240 | 190 | 180 | 160 |
| | 15 | 230 | 195 | 170 | 140 |
| | 16 | 230 | 190 | 160 | 140 |

Although effects on feed intake and milk production by aloe supplements have been observed elsewhere, the inventor's figures as shown in Table 3 do not show significant effects in this aspect, over a range from 0 to 10 ml per day.

TABLE 3
Effect of Tonic on Feed intake, Milk
yield, Milk fat, protein and lactose

| Tonic | Feed | Milk Yield | Milk | Milk | Milk |
|---|---|---|---|---|---|
| ml/day | Intake | Litres | Fat % | Protein % | Lactose % |
| zero | 45 | 36 | 3.7 | 3.2 | 4.5 |
| 1 | 45 | 38 | 3.8 | 3.4 | 4.6 |
| 5 | 45 | 37 | 3.8 | 3.4 | 4.6 |
| 10 | 45 | 38 | 3.8 | 3.4 | 4.6 |

In addition there is anecdotal evidence (as letters from farmers) that calf scours are suppressed and calf health is improved if calves are given oral or in-food treatments of manuka and aloe tonic according to the invention.

### EXAMPLE 4

### In-Vitro Tests

1. Bacterial growth inhibition. This was carried out by an independent laboratory with a standard procedure (variation of the Microtitre Broth Dilution procedure) to determine 50% inhibition of growth (MIC₅₀) Staph=Staphylococcus aureus. Strep=Streptococcus uberis. The results show moderate direct antibacterial activity.

TABLE 4

| | Staph MIC₅₀ at 12 h | Strep MIC₅₀ at 12 h |
|---|---|---|
| First run: 19 Sep 2007 | | |
| Sample A: | Dil: 1:4 | Dil: 1:4 |
| Sample B: | Dil: 1:8 | Dil: 1:16 |
| Second run: 2 Oct 2007 | | |
| A: pH = 6 | Dil: 1:27 | Dil: 1:27 |
| A: pH = 4 | Dil: 1:27 | Dil: 1:27 |
| B: pH = 6 | Dil: 1:27 | Dil: 1:9 |
| B: pH = 4 | Dil: 1:51 | Dil: 1:51 |
| Third run: 30 Oct 2007 | | |
| C: pH = 4 | Dil: 1:4 | Dil: 1:4 |
| C: pH = 7 | Dil: 1:2 | Dil: 1:4 |

### 2. Monocyte Immune Stimulation Assay.

This assay measures the ability of a substance to alter production of an inflammatory cytokine (TNFα), using human monocytes. In the presence of a bacterial cell wall component (a lipopolysaccharide) that simulates a gram-negative bacterial infection, significant stimulation of cytokine was noted as a result of exposure to B at pH=6. It is concluded that the composition of the invention has a useful effect on the immune system, but further work is needed. These in-vitro tests support the clinical observations made at the whole-cow level.

### Acute Mastitis.

A mixture of honey and aloe vera according to the present invention may be at least partially neutralised before use, so that the pH is around 6 rather than about 3-4, and introduced through the teat canal as an intramammary treatment for mastitis on preferably a twice-daily basis for three to five days. First, the affected quarter is cleaned and then stripped (emptied by usually hand-milking) and then about 20-30 ml of a 1:10 composition is introduced and massaged about the udder. Clinical resolution usually follows. This treatment has the advantages (a) that milk from other quarters need not be withheld from the bulk milk vat since no antibiotic has been used, and hence antibiotic resistance by the pathogens is not caused or fostered. Note that there is no reason to limit treatment to aloe plus honey only, since an antibiotics course (using a standard intramammary antibiotic course) may be used as well. No cases of gangrenous mastitis (a severe type) have as yet been attempted.

### General Conclusions:

From Table 3 and from observations: The manuka honey and aloe food supplement or tonic does not adversely affect health and production of dairy cattle. The tonic does not adversely affect any of the standard measures of animal health. Composition of milk, feed intake, and milk yield remains normal, in normal animals, on feeding the tonic. Yet the tonic administered orally or as a food supplement has a significant beneficial effect on animals affected with chronic mastitis and it is legitimate to assume that other chronic diseases affecting cows would also be at least partially alleviated by its use.

As evidenced by FIG. 1, and Tables 1 and 2: The manuka honey and aloe food supplement/tonic in a sufficient dose decreases the somatic cell count (SCC) in dairy cattle for a subsequent period. A low somatic cell count indicates absence of infection. This indicates that the tonic decreases the susceptibility to infection. Since the quality of milk from cows treated with the tonic is higher, the tonic provides a double advantage in decreasing milk losses and increasing quality of milk.

Table 4 confirms that there are effects inherent in the composition, but the particular tests used may not reflect the improved resistance to disease on a whole-cow basis over an extended period. Effects, seen over some months, such as better visual alertness combined with better response to stress and infection have been noted by dairy farmers but are harder to quantify.

### Variations

Although all trials to date have used manuka honey, the actual UMF content as supplied is unspecified and may be below average since there are commercial demands elsewhere for high UMF honeys. Future experiments will test the effectiveness of manuka honey components with known levels of the UMF factor. Further, future experiments will test other honeys.

Quantitative proof of synergism, such as experiments in which some members of a population under treatment are given a placebo, others are given an accepted treatment (e.g. a commonly used antibiotic) while the remainder are treated with the composition of the invention or components thereof is not yet available.

Further experimentation is also needed in relation to aloe vera/honey treatments before or during the drying-off period which in seasonal dairy farming occurs at the onset of winter.

Use of the composition in the thoroughbred industry, by feeding it to breeding mares on a long-term basis at about the standard cow dose, may help overcome the breeding problems often experienced in this industry.

### INDUSTRIAL APPLICABILITY AND ADVANTAGES

Prophylaxis: The food supplement or tonic made of aloe vera and honey appears to have general health benefits in a dairy herd apart from specific beneficial effects as quantified by the somatic cell count. Indeed, one farmer claims that many cows tend to come into heat on or near the same day. Note that since the composition is “Organic” according to the relevant industry definitions, no special care to avoid introducing the milk into the human food chain is required. For instance, there is no antibiotic residues to cause allergies or to promote antibiotic resistance after ingestion.

More generally the composition of the invention may be used to improve the immune status or capability of any mammal or group of mammals, such as a group affected by chronic illness.

Therapy: Oral treatment to mitigate the effects of chronic mastitis by means of a short course of a higher dose of the composition of the invention is successful. Absent this invention, overcoming chronic mastitis in recalcitrant cows may be difficult and the cows will have to be culled in order to avoid having a reservoir of infection within the herd.

Apart from the farming applications referred to previously, the product can be sold for use as a human beverage, when some of the additives such as ginger will enhance the distinctive yet pleasant flavour of the mixture.

Finally, it will be understood that the scope of this invention as described by way of example and/or illustrated herein is not limited to the specified embodiments. Where in the foregoing description, reference has been made to specific components or integers of the invention having known equivalents, then such equivalents are included as if individually set forth. Those of skill will appreciate that various modifications, additions, known equivalents, and substitutions are possible without departing from the scope and spirit of the invention as set forth in the following claims.

1-18. (canceled)

19. A mixture of aloe vera and honey for use primarily as a food supplement or tonic, characterised in that the composition is sold for distribution then use as a food supplement or tonic for animals including humans.

20. A mixture as claimed in claim 19, characterised in that the honey is a predominantly manuka honey.

21. A mixture as claimed in claim 19, characterised in that the mixture is formulated as a concentrate for use in dairy farming, for the suppression and control of chronic mastitis.

22. A mixture as claimed in claim 19, characterised in that the composition is in a non-concentrated form ready for oral administration.

23. A mixture as claimed in claim 19, characterised in that the composition is in a form ready for use by intramammary administration suitable for the treatment of mastitis in cows.

24. A mixture as claimed in claim 19, characterised in that the mixture comprises a composition including on a weight for weight basis the following ingredients:

| | |
|---|---|
| Manuka honey | from 6% to 10%, |
| Aloe vera powder | from 79% to 98.7% |
| Sodium benzoate | from 0.1% to .5% |
| Potassium sorbate | from 0.1% to .5% |
| Ginger powder | from 2% to 8% (flavouring) |
| pH adjustment: | citric acid 0.1% or apple cider vinegar |
| | 0.1%, to achieve a pH in the range of |
| | 3.5 to 5. |

25. A mixture as claimed in claim 24, characterised in that the mixture also includes tea tree oil or extracts thereof.

26. A mixture as claimed in claim 24, characterised in that the mixture also includes garlic powder or extracts thereof.

27. A mixture as claimed in claim 24, characterised in that the mixture also includes Azadirachta indica (neem) extracts.

28. A mixture as claimed in claim 24, characterised in that the mixture also includes Curcuma longa (turmeric) extracts

29. A mixture as claimed in claim 24, characterised in that the mixture is concentrated by dehydration to from 2:1 to about 20:1

30. A method for raising the level of immunity in a group of animals, comprising the repeated administration of a mixture as claimed in claim 21 orally or in the food to the animals for a period.

31. A method as claimed in claim 30, for reducing the incidence of chronic bovine mastitis, comprising the repeated administration of the mixture orally or in the food to an entire herd during the lactation season.

32. A method as claimed in claim 30, characterised in that the preferred amount of the mixture is between about 1 ml and 5 ml per cow per day (the quantity is expressed as the concentrate).

33. A method for treating chronic bovine mastitis, comprising the administration of a mixture, as claimed in claim 21 orally or in the food as several large doses over a short space of time.

34. A method for treating chronic bovine mastitis as claimed in claim 33, comprising the administration of three doses of 30 ml (expressed as concentrate) to an affected cow, given orally over one week.

35. A method for treating bovine mastitis, comprising the repeated intramammary administration of a mixture as claimed in claim 21 after stripping the udder of fluid.

36. A method for treating bovine mastitis, comprising the repeated intramammary administration of a mixture as claimed in claim 21, plus an effective amount of an intramammary antibiotic after stripping the udder of fluid.

37. A mixture as claimed in claim 20, characterised in that the mixture is formulated as a concentrate for use in dairy farming, for the suppression and control of chronic mastitis.

38. A mixture as claimed in claim 20, characterised in that the composition is in a non-concentrated form ready for oral administration.
